(19) United States
(12) Patent Application Publication
(10) Pub. No.: US 2003/0124659 A1
(43) Pub. Date: Jul. 3, 2003

(54) Multiplexing method

(76) Inventor: David James Graves, Devon, PA (US)

Correspondence Address: WEBB ZIESENHEIM LOGSDON, ORKIN & HANSON, P.C., 700 Koppers Building, 436 Seventh Avenue, Pittsburgh, PA, 15219-1818, US

(21) Appl. No.: 10/165,186

(22) Filed: Jun. 7, 2002

Related U.S. Application Data
(60) Provisional application No. 60/296,513, filed on Jun. 7, 2001.

(51) Int. Cl. C12P 21/06

(52) U.S. Cl. 435/69.1

(57) ABSTRACT

A “process of elimination” method of creating mixtures of labeled particles, allowing the particles to interact, documenting particle interactions or lack thereof, and collating information derived from more than one experiment, with each experiment combining particles from one or more mixtures with other particles.

### CROSS REFERENCE TO RELATED APPLICATION

This application claims the benefit under 35 U.S.C. §119(e) of U.S. Provisional Patent Application Serial No. 60/296,513, filed on Jun. 7, 2001, which is incorporated herein by reference in its entirety.

### BACKGROUND OF THE INVENTION

1. Field of the Invention

The field of this invention relates to methods of performing multiple chemical or biochemical experiments in the same physical location while maintaining the ability to assign results to individual experiments.

2. Description of Related Art

Currently, if an individual in a research or industrial setting wants to determine if four particle types of one group, say Group A (particle types A1, A2, A3, A4), interact with four particle types of another group, say Group B (particle types B1, B2, B3, B4), he or she would usually perform 16 separate experiments wherein one particle type of Group A is mixed with one particle type of Group B for all possible combinations: A1+B1; A1+B2; A1+B3; A1+B4; A2+B1; A2+B2 . . . A4+B4. After the two particles are mixed together some detection method is typically employed to determine whether or not the two particles interact. As a matter of nomenclature, the above-described experiments are referred to as simplex experiments, because only one particle type/particle type combination is examined at any one time. However, because it is often technically desirable to examine the interactions of more than one particle type with more than one particle type, for example with complex substances for which all the interactions are to be identified, a need remains for assays which accomplish multiplexing, namely, interaction assessment of more than one particle type from a group with more than one particle type from a second group. The simplest multiplex experiment would be to mix one particle from one group with two from another, as in A1+B1+B2. An experimental procedure that has unlimited multiplexing capabilities would be one in which any number of desired particles can be mixed together and all possible combinations of interactions between all particles can be distinguished.

Heretofore, limitations in detection technologies have been responsible at least, in part, for limited innovation in the multiplexing area. Fluorescence, for example, has broad emission spectra. Consequently, mixing two or more different colored fluorophores together can yield light having peaks that are difficult to differentiate. Recent advances in labeling technology, such as quantum dots and quantum dot embedded beads, may enable multiplexing by labeling all particle types with unique labels. Despite much scientific effort, however, it is still not possible to create unique labels for each particle type when more than a few particle types are admixed. For this and other reasons, a need remains for a multiplexing method which makes possible multiplexing interaction testing which is effective even with the limited labeling technologies available at this writing.

If such technology can be perfected then nearly unlimited multiplexing capabilities would be possible. However, such technologies are not yet perfected.

The present invention is a method to create mixtures of labeled particles, and a method of using them that enables significant multiplexing while using even simple labeling technologies.

### SUMMARY OF THE INVENTION

In order to meet this need, the present invention is a “process of elimination” method of creating mixtures of labeled particles, allowing the particles to interact, documenting particle interactions or lack thereof, and collating information derived from more than one experiment, with each experiment combining particles from one or more mixtures with other particles. The inventive method inheres in part in choosing which mixtures to make in order to minimize the number of experiments that must be performed in order to ascertain which particles interact. As a part of the “process of elimination” approach, the present invention harnesses negative interaction results in addition to positive interaction results so as to minimize the overall number of tests required to complete the investigation. The present invention also provides for a computer system for analyzing the results of each experiment and for using the information derived from the experiments to substantiate comprehensive conclusions regarding particle interaction.

### DESCRIPTION OF THE PREFERRED EMBODIMENT(S)

The present invention is a “process of elimination” method of creating mixtures of labeled particles, allowing the particles to interact, documenting particle interactions or lack thereof, and collating information derived from more than one experiment, with each experiment combining particles from one or more mixtures with other particles. The inventive method inheres in part in choosing which mixtures to make in order to minimize the number of experiments that must be performed in order to ascertain which particles interact. As a part of the “process of elimination” approach, the present invention harnesses negative interaction results in addition to positive interaction results so as to minimize the overall number of tests required to complete the investigation. The present invention also provides for a computer system for analyzing the results of each experiment and for using the information derived from the experiments to substantiate comprehensive conclusions regarding particle interaction.

In order to gain an understanding of the present invention, it is helpful to start with a simple, yet realistic, scenario using the following definitions.

Particle: a physical object including but not limited to various chemical species such as DNA fragments, peptides, proteins, lipids, cells or other discrete physical objects potentially susceptible of physical or chemical binding or any other chemical, physical or electrical interaction.

Particle type: two or more particles substantially equivalent in their interactional characteristics.

Label: an artifact of any technology, associated with a particle, which enables detection of the particle. Examples including, but are not limited to, fluorescence, luminescence, radio isotopes, quantum dots, dyes, etc.

Label type: two or more labels substantially equivalent in their interactional characteristics, so that labels, which can be distinguished, would be of different types.

In this initial scenario, consider two groups of particles, namely, Group A and Group B. Group A contains the particle types A1, A2, A3, A4; Group B contains the particle types B1, B2, B3 and B4. Suppose one wanted to determine if any particles from Group A bind to any particles from Group B, and specifically which particle types from each group bind together. Further suppose that, in fact, A1 and B1 interact by binding together as do A4 and B4, though this information is not known a priori.

With the present invention, Group B particles could be spotted onto a glass slide in known positions. Since there are only four compounds, they could be spotted close to the corners of a glass slide. Suppose that B 1 is spotted close to the upper left (UL) corner, B2 close to the upper right corner (UR), B3 close to the lower left (LL), and B4 close to the lower right (LR). As will become apparent in view of the following explanation, only two slides would be needed to specify which interactions occur between particles of Groups A and B.

Group A particles could be divided into two samples each, not necessarily of equal quantity or concentration but equal for the purposes of this initial illustration. Thus, there would be a total of eight samples, two for each of the four particle types of Group A. Each sample could be labeled with either green fluorescence or yellow fluorescence such that no two samples of one particle type have the same label type. For example, one sample of A1 particle types would be labeled with green fluorescence and the other sample of A1 particle types would be labeled with yellow fluorescence. The same would be true for A2, A3, and A4 particle types, yielding eight labeled samples with no two samples of the same particle type having the same label type.

Then, two mixtures each are created from the eight samples. The mixtures could differ from each other in any or all of the following characteristics: combination and number of particle types, concentration of one or more particle types, the type of label employed with one or more particle types, the number of label types employed with one or more particle types, and the number of label particles used to label an individual particle. However, for simplicity in this illustration the two mixtures would be comprised of A1, A2, A3, and A4 particle types such that the concentration of A1 in the first mixture would be equal to the concentration of A1 in the second mixture. Furthermore, Al would be labeled with only one label type in each mixture—i.e., for each mixture Al would be taken from only one of the two samples of A1, and each sample of A1 is labeled with either green or yellow fluorescence, but not both. The same would be true for A2, A3, and A4. For discussion purposes, then, the two mixtures differ from each other only in the choice of label type for each particle type. Even so, there would be 16 possible mixtures including particle types from Group A and Group B, as shown in Table 1. Note in the table below that “1” will be used for yellow fluorescence, “0” will be used for green fluorescence, and “M1” means “mixture 1,” “M2” means “mixture 2,” etc.

TABLE 1

| | M1 | M2 | M3 | M4 | M5 | M6 | M7 | M8 | M9 | M10 | M11 | M12 | M13 | M14 | M15 | M16 |
|---|---|---|---|---|---|---|---|---|---|---|---|---|---|---|---|---|
| A1 | 1 | 1 | 1 | 1 | 1 | 1 | 1 | 1 | 0 | 0 | 0 | 0 | 0 | 0 | 0 | 0 |
| A2 | 1 | 1 | 1 | 1 | 0 | 0 | 0 | 0 | 1 | 1 | 1 | 1 | 0 | 0 | 0 | 0 |
| A3 | 1 | 1 | 0 | 0 | 1 | 1 | 0 | 0 | 1 | 1 | 0 | 0 | 1 | 1 | 0 | 0 |
| A4 | 1 | 0 | 1 | 0 | 1 | 0 | 1 | 0 | 1 | 0 | 1 | 0 | 1 | 0 | 1 | 0 |

It is helpful to consider what information could be ascertained by pouring mixture 1 on a glass slide containing the four Group B particles as described above, and mixture 2 on another identical glass slide. If mixture 1 was poured onto the glass slide, incubated, and washed, the UL and LR would fluoresce yellow because A1, labeled with yellow fluorescence, would bind to B1 and A4, also labeled with yellow fluorescence, would bind to B4. The information ascertained from this exercise is that B1 and B4 interact with at least one particle from Group A, and B2 and B3 do not interact with any particles from Group A. When mixture 2 is placed on an identical glass slide containing B particle types, as described above, the UL position would fluoresce yellow and the LR position would fluoresce green. From the combined results from the two slides, one ascertains that B4 interacts with A4, B1 interacts with at least one of the remaining three particles of Group A, and B2 and B3 do not interact with any particles from group A. Thus, some mixtures and reactions will provide more information than others, and choosing wisely can substantially increase the information received from one experiment. For example, in Table 1, mixtures 11 and 13 can be used with two slides to yield enough information to ascertain completely which particles interact. Mixture 11 is used with one slide (S1) and mixture 13 is used with the other slide (S2). On S1, UL would fluoresce green and LR would fluoresce yellow, indicating that B1 interacts with either A1 or A3 but not A2 or A4, B2 and B3 interact with no group A particles, and B4 interacts with either A2 or A4 but not A1 or A3. One could then use this information to evaluate S2, where the UL position would fluoresce green and the LR position would fluoresce yellow. From S2 one could ascertain that B1 interacts with either A1 or A2 and B4 interacts with either A3 or A4. However, from S1, one would know that B 1 does not interact with A2 and B4 does not interact with A3. Combining the information from S1 and S2 would allow one to ascertain that B1 interacts with A1, and B4 interacts with A4, with no other interactions. Negative information, namely, information about which particles do not interact, would be used to limit the number of subsequent experiments required to ascertain all interacting pairs between Group A and Group B particles. The present invention thus uses negative information from test results to derive conclusions from a group of experiments where each experiment, by itself, might not yield conclusive information. Applicant is aware of no detection method which uses negative information to limit the number of experiments that must subsequently be performed in order to determine comprehensive interaction information.

In the illustration just provided, two label types were used-green and yellow fluorescence. This is referred to as a Binary labeling system, or more generally, as a Multinary labeling system. It is not necessary that a Binary labeling system be used. A Monary (only one label type) or Multinary (two or more label types) labeling system can be used. In the preferred embodiment, a Binary labeling system is used because it would employ standard labeling and detecting technologies. However, if more label types can be easily distinguished, then it is preferable to use more label types.

The above illustration explains how mixtures of labeled particles can be used in the present multiplexing method, which is a process of elimination protocol. The number of experiments needed to ascertain which particles interact can be reduced, in the present scenario, from 16 (when simplex evaluations are used) to two. The above illustration helps explain how the present invention can be used to multiplex, but it does not explain all aspects of the present invention. For example, this first simple illustration does not explain how to choose which two of the 16 possible mixtures of labeled particles to use.

One aspect of the present invention is a method to choose which mixtures of labeled particles to create in order to minimize the number of experiments required to determine which pairs of molecules interact. In the above introductory scenario, no particle types of Group A interacted with more than one particle type of Group B. Instead, A1 interacted with B1 only, and A4 interacted with B4 only. In reality, one cannot be assured that there will not be multiple interactions between one particle type of one group and the particle types of another group. The present invention can be used when multiple interactions occur. However, initially, it is easier to understand the present invention by assuming that each particle type from one group interacts with no more than one particle type from the other group.

Assuming that each particle type from one group interacts with no more than one particle type from the other group, the minimum number of experiments required to determine all possible interactions is defined by the equation Logₓ(A)=Z, where x=the number of label types, A=the number of labeled particle types, and Z=the minimum number of experiments required. If Z is a not a whole number, it must be rounded up to the next whole number. For example, if A=11 and x=3, Log₃(11)=2.18, which would indicate the minimum number of experiments is 3. Sometimes less than this minimum number of experiments can be performed, so really in the broadest sense the equation is really Z=y(LogₓA) in which y=0.1-1.0. The possible variability of y is explained further below.

The variable “A” was not picked at random. In the illustrative example above, Group A particles were labeled and screened against Group B particles. In the equation Logₓ(A)=Z, “A” is analogous to “Group A.” Note that the number of experiments necessary is not dependent on how many particles “A” (Group A) will be screened against (Group B). Thus, in the illustrative example above, Group B could have consisted of any number of particles and still only two glass slides would have been needed. Of course, there is a physical limitation to how many particles could have been spotted on a glass slide. Physical limitations may require more glass slides or reaction substrates generally, but the number of mixtures required is still the same (although a larger quantity of each mixture may be needed because a sample of each mixture may be required for multiple slides). One skilled in the art will also recognize that cross reactions between particles in Group A could require one to limit the number of particle types in Group A. If one is forced to limit the number of particle types in Group A for a set of experiments, the equation Logₓ(A)=Z, nevertheless, correctly indicates the number of mixtures required for however many particle types are finally employed in Group A, though multiple sets of Group A particles may be necessary. If more than one set of Group A particles is necessary, the equation Logₓ(A)=Z must be used to determine the minimum number of experiments required for each set of Group A particles. The equation, then, correctly indicates the minimum number of experiments required for each set of Group A particles to be screened against a set of Group B particles. To repeat, this is true as long as one particle type from Group A interacts with, at most, one particle type from Group B. Later in this specification, we will relax this restriction and discuss how the present invention can be used to determine particle interactions even when a particle type from Group A interacts with more than one particle type in Group B.

After the minimum number of experiments is calculated, one may use more experiments if so desired, to reduce the false positive and false negative errors. Preferably, however, the minimum number of experiments is used. The next step is to determine which mixtures to use in the minimum number of experiments. The mixtures must not employ any degenerative codes, which is explained next. To ensure no degenerative codes are employed, create a table with compounds in the row headings and mixtures in the column headings, as used in the first scenario above. However, the table should allow only for the number of mixtures that equal the minimum number of experiments possible. For example, if four compounds are to be labeled with two labels, then the minimum number of experiments would be Log₂ (4)=2, and the table should be set up as in Table 2 below.

TABLE 2
TABLE SET UP TO DETERMINE MIXTURES FOR
X = 2 AND A = 4

| M | M |
|---|---|
| 1 | 2 |

A1
A2
A3
A4

Fill the cells in each row with either a “0,” to indicate one label, or a “1,” to indicate the other label. When doing so, ensure that each pattern is used only once. For example, fill the first row with the pattern 0, 1, as shown in Table 3.

TABLE 3
FILLING FIRST ROW WITH PATTERN 0,1

| | M | M |
|---|---|---|
| | 1 | 2 |
| A1 | 0 | 1 |
| A2 | | |
| A3 | | |
| A4 | | |

Now that the pattern 0,1 is used, fill the remaining rows without ever using 0,1 again or without using any pattern twice. If the next pattern used is 1,0, then there are only two patterns to choose from for the remaining two rows—0,0 and 1,1. If 0,0 is used for the third row then 1,1 must be used for the fourth row, and the resulting table is shown in Table 4.

TABLE 4
FILLING ALL ROWS WITH UNIQUE PATTERNS
TO DETERMINE MIXTURES

| | M | M |
|---|---|---|
| | 1 | 2 |
| A1 | 0 | 1 |
| A2 | 1 | 0 |
| A3 | 0 | 0 |
| A4 | 1 | 1 |

If a horizontal pattern is used more than once, it is said that the mixtures contain degeneracy or contain degenerative patterns for the purposes of this document. If even one degenerative pattern is used, one cannot be assured that the experiments will distinguish all pairs of particles that interact. This method of determining the composition of distinguish all pairs of particles that interact. This method of determining the composition of mixtures for the minimum number of experiments works regardless of how large x and A are.

In the case where Z has been rounded up, there will be multiple choices for the pattern that fills the last row of the table. For example, if A=11 and x=3, then Logₓ(A)=2.18, and the minimum number of mixtures is 3. If “0” is used for one label type, “1” is used for a second label type, and “2” is used for a third label type, then the mixtures could be determined as in Table 5.

TABLE 5
MIXTURE DETERMTNATION FOR A = 11 AND X = 3

| | M | M | M |
|---|---|---|---|
| | 1 | 2 | 3 |
| A1 | 0 | 0 | 0 |
| A2 | 0 | 0 | 1 |
| A3 | 0 | 0 | 2 |
| A4 | 0 | 1 | 0 |
| A5 | 0 | 1 | 1 |
| A6 | 0 | 1 | 2 |
| A7 | 1 | 0 | 0 |
| A8 | 1 | 0 | 1 |
| A9 | 1 | 0 | 2 |
| A10 | 1 | 1 | 0 |
| A11 | 1 | 1 | 1 |

Any row pattern above could be changed to any other pattern that has not yet been used. In fact, any number of the row patterns in Table 5 could be changed to patterns that have not yet been used as long as any single pattern is not used twice. Extra patterns exists for the last row of Table 5 because the number of particles in Group A is fewer than the maximum number that can be distinguished with three mixtures using three label types. The maximum number can be calculated by (x) raised to the number of mixtures. In Table 5, the number of mixtures is 3, so the maximum number is (3)3=27.

Also, extra mixtures can be added to increase the number of patterns available so that more patterns are available than are needed. Doing so can reduce false positives and false negatives by choosing patterns that reduce the number of particles that contain the same label in each mixture, as long as no patterns is used twice. Also, multiple sets of experiments can be performed to reduce false positives and false negatives using mixtures for one set of experiments that differ from those used in another set of experiments.

To develop this idea further, it is time to relax our assumption that a single particle type from one group interacts with no more than one particle type of the other group. It may be the case that particles from one group can interact with more than one particle from another group. When this happens, it is possible that after the “minimum” number of experiments is accomplished, one skilled in the art will be able to determine that a compound from one group interacts with more than one particle from the other group, but be unable to determine exactly which ones. This will occur when the detecting step is not capable of distinguishing multiple label types. This can be illustrated if we modify the first above illustration as follows. Group B particles will still have four particle types, B1, B2, B3 and B4, and would be arrayed on a glass slide the same as described above. Group A particles will have 11 particle types. Instead of having two label types, the new scenario, called Scenario 2, will have four label types which are green, yellow, red, and blue fluorescence, indicated by 0, 1, 2, 3, respectively. In Scenario 2, we will assume the detection machinery is incapable of distinguishing three or more label types, when detected together, from any other combination of three or more label types. A detection that cannot distinguish between individual label types will be called “fuzzy.” However, in Scenario 2, the detection machinery is capable of distinguishing any two label types when detected together. Using the method described above to determine the minimum number of experiments, we can construct a table as follows:

TABLE 6
MIXTURES FOR SCENARIO 2

| | M | M | M |
|---|---|---|---|
| | 1 | 2 | 3 |
| A1 | 0 | 0 | 0 |
| A2 | 0 | 0 | 1 |
| A3 | 0 | 0 | 2 |
| A4 | 0 | 0 | 3 |
| A5 | 0 | 1 | 0 |
| A6 | 0 | 1 | 1 |
| A7 | 0 | 1 | 2 |
| A8 | 0 | 1 | 3 |
| A9 | 0 | 2 | 0 |
| A10 | 0 | 2 | 1 |
| A11 | 0 | 2 | 2 |

In Scenario 2, assume that A1, A2, and A3 bind to B1. Also assume that A4, A5, and A6 bind to B4. Each mixture would be poured on a slide, incubated, washed, and the four spots, UL, UR, LL, and LR would be subjected to the detection machinery. Since only B1 and B4 bind particles from Group A, only the UL and LR spots will fluoresce. Table 7 indicates what is detected for UL and LR. Note that fuzzy indicates a mixture of three or more fluorescent colors. Also note that “S1” is the glass slide on which mixture 1 was poured, incubated, and washed. Likewise, “S2” corresponds to the slide used for mixture 2 and “S3” corresponds to the slide used for mixture 3.

TABLE 7
COLORS DETECTED ON EACH POSITION OF EACH
SLIDE FOR SCENARIO 2

| | S1 | S2 | S3 |
|---|---|---|---|
| UL | Green | Green | Fuzzy |
| LR | Green | Green & Yellow | Fuzzy |

It is helpful to analyze one spot at a time for all three slides. Table 8 indicates the information that can be ascertained about B 1 from each slide for UL independently, and then using the cumulative information from the previous slide(s) to narrow the possibilities for the current slide.

TABLE 8
INFORMATION ABOUT WHICH COMPOUNDS BIND
B1 (UL) FROM ALL THREE SLIDES FOR SCENARIO 2

| | S1 | S2 | S3 |
|---|---|---|---|
| Independent | A1-A11 | A1-A4 | At least three, one from each of the |
| | | | following groups: (A1, A5, A9) (A2, |
| | | | A6, A10) (A3, A7, A11) (A4, A8) |
| Cumulative | A1-A11 | A1-A4 | At least three from the group |
| | | | (A1, A2, A3, A4) |

Table 9 similarly displays the results for B4 (LR).

TABLE 9
INFORMATION ABOUT WHICH COMPOUNDS BIND B4 (LR)
FROM ALL THREE SLIDES FOR SCENARIO 2

| | S1 | S2 | S3 |
|---|---|---|---|
| Independent | A1-A11 | At least one from | At least three, one from each of the |
| | | each group: (A1-A4) | following groups: (A1, A5, A9) (A2, |
| | | & (A5-A8) | A6, A10) (A3, A7, A11) (A4, A8) |
| Cumulative | A1-A11 | At least one from each | At least three, one from each of the |
| | | group: | groups (A1, A5) (A2, A6) (A4, A8) (A3, |
| | | (A1-A4) & | A7) such that at least one from each of |
| | | (A5-A8) | the groups (A1-A4) & (A5-A8) are |
| | | | included. |

One skilled in the art could devise experiments utilizing the information tabulated in Tables 8 and 9 definitively to determine which Group A compounds bind to B1 and B4. Notice that the present invention narrows the search even when fuzzy detections are recorded. Thus, the present invention is valuable even when multiple interactions between particle types yield information that is not definite.

In fact, when the possibilities are small that multiple interactions will occur, it may be more economical to assume that no multiple interactions occur and perform the minimum number of experiments according to the equation Logₓ(A)=Z. If any fuzzy results do occur, a limited number of subsequent experiments can be performed to clearly identify which particles interact.

One skilled in the art will also realize that if the likelihood of any interactions at all are small, it may be economical to perform fewer than the minimum number of experiments according to the equation Logₓ(A)=Z. Doing so may increase the chances of receiving fuzzy data, but the number of experiments required to achieve conclusive results may actually be fewer because the experiments that are necessary to resolve the fuzzy data may be few and involve few compounds.

The present invention can be used when only a single label type is employed, which is termed a Monary label system. In that case, the combinations of labeled particles would differ by containing different types of Group A particles. It is important to expound on the current invention as it relates to Monary labeling systems to understand the flexibility of the present invention. We will consider another scenario, Scenario 3, that has eight particle types in Group A (A1, A2 . . . A8) and five particle types in Group B (B1, B2 . . . B5). We shall also assume that A1 binds to B1 and A5 binds to B5. Group B particles are attached to beads, which are packed in a column. Each column contains only one particle type from Group B. The columns will be referred to as C1-C5 and the number corresponds with the particle type from Group B. Group A particles are labeled with radioactivity and mixtures are made from them. Because there is only one label type, a table is not necessary to display the mixtures. Using “M1” to mean mixture 1, as it was used previously, the following mixtures could be made: M1 contains A1-A4; M2 contains A5-A8. The volume of each mixture is large enough so that an adequate portion of each mixture can be run through each column. First, a sample of M1 is passed through each of the five columns and then each column is washed. Each column is checked for radioactivity. In this scenario, C1 will be radioactive, but none of the others will be. This data would indicate B1 binds to at least one particle in M1, but no other Group B particles bind to A1, A2, A3, or A4. Cl could then be stripped of Group A particles. M2 could then be added to each column. C5 would then be radioactive, but no other column would be. This data would indicate that B5 binds to at least one of the particles in M2, while the other Group B particles do not bind A5, A6, A7, or A8. Fuzzy data is produced for B1 and B5, but concrete negative information is produced for all possible combinations between B2, B3, and B4 with all Group A particles. C1 could be further tested with two other mixtures, M3 and M4. M3 would contain labeled A1 and A2 while M4 would contain labeled A3 and A4. One skilled in the art will realize that successive steps that add mixtures containing half of the particles remaining to be screened will eventually produce conclusive results. A similar set of experiments will resolve the fuzzy data for C5. One note of caution is pertinent. When the last two particles in question are individually added to the column, it is important to test both particles just in case both particles bind.

The present invention is susceptible to widespread modification without departing from the scope of the invention. Glass slides or beads are not the only substrates useful to conduct the assays described herein, which can be performed on any surface or in any configuration. Also, Group B particles could be labeled with one or more label types and be detected as well. If that were done, the particles could be passed through a flow cytometer and detected in conjunction with Group A particles. Another method to identify Group B particles would be to use well plates where each well contains only one Group B particle or more than one Group B particle which can be identified by a label. Another method to identify Group B particles is to attach them to different sizes of beads which can be identified by microscopy, flow cytometry, size-exclusion chromatography, filtering, etc. Other methods include: radio isotopic labeling, quantum dot labeling, labeling with dyes, luminescence, labeling by attachment to a device that emits radio frequencies, labeling with magnetized particles, or any combination of these labeling methods. Furthermore, other label types are possible. For example, Group B particles can be attached to secondary particles which can be identified by binding to yet other particles. DNA and biotin are two examples of secondary particles which can be identified by binding to yet other particles, namely, complementary DNA and streptavidin, respectively. The secondary particles could be attached to another substrate, such as beads, glass particles, micro metallic rods, etc., to which the secondary particles are also attached. In this patent “label type” includes molecular or radio isotopic labels alone or any combination of them with secondary particles to which other identifying particles bind or substrates to which secondary particles or the labels themselves bind. This applies to both Group A and Group B particles, and one skilled in the art can devise many combinations of labeling schemes for each group to increase the power of the present invention. The power of the present invention is determined by how many unique labels can be simultaneously detected and differentiated. One skilled in the art will also recognize that the value of the present invention increases dramatically with the number of label types employed and with the number of particles to be screened. At the same time, one skilled in the art will be able to practice the above-described invention with imprecision and still fall within the scope of the invention. For example, even if at least 10% of the combinations of samples from two groups, but not less than two, are free from degenerative patterns, the present invention may be meaningfully practiced. Ultimately, then, the operative formula is Z=y(LogₓA) in which Z is the minimum number of group sample combinations to be combined; A is the largest number of particle types in any group; x is the number of label types, and y=0.1 to 1.0. The lower values of y suffice when significant degeneracy can be tolerated and the higher values of y apply when a non-degenerate set of combinations is desired.

It is also possible to increase the power of the present invention by using multiple detection technologies on the same sample. For example, one could detect fluorescence with one detector, radiation with another, size of a bead with yet another, and magnetic field with still another detector. If this is accomplished on the same experimental sample, it would be equivalent to detecting all simultaneously while retaining the ability to differentiate them.

The preferred embodiment of the present invention:

1) uses a Binary labeling system;

2) has all particle types in each mixture;

3) has substantially the same concentration of particle types in each mixture;

4) employs only one label type per particle type in each mixture;

5) uses substantially the same number of labels per particle in each mixture;

6) reacts each mixture with single samples of collections of other particles, each sample being substantially the same as other samples, and which may or may not be labeled;

7) uses a method to detect labeled particles and a method to correlate them with sample particles, either by detecting sample particles because they are labeled or by known location of the sample particles; and

8) uses information from preceding experiments to interpret data from subsequent experiments.

However, the present invention may differ from the preferred embodiment in any or all of the characteristics listed above. One can adopt any or all of the following variations:

1) use a Monary or Multinary labeling system;

2) have less than all particle types in one or more mixtures;

3) have different concentrations of one or more particle types in one or more mixtures;

4) employ multiple label types for one or more particle types in one or more mixtures;

5) use different number of label particles per particle type for one or more particles in one or more mixtures;

6) use samples of collections of other particles which are different from other samples in composition of particle types, concentration of particle types, location of particle types, or label of particle types;

7) use different methods to detect labeled particles and different methods to detect sample particles; and

8) use or not information from some experiments to interpret data from other experiments.

The present invention also provides for a computer to combine information from multiple experiments. In the illustrative examples using a glass slide, a computer can record the color of each location automatically. From a database about each slide, the computer can narrow the possibilities about which particles from one group interact with particles of another group. The computer can then read other slides and can further narrow the possibilities of interacting pairs until conclusive results are obtained.

As is apparent from the above description, the present invention can be used to screen compounds from one group against those in another group to identify which ones interact. This is important in pharmaceutical research where large compound libraries are screened against targets in an effort to identify potential drugs. Many of these experiments are designed to detect binding interactions between the targets and members of the compound library. The present invention can also be used to determine proteins that interact with each other. Random libraries of proteins can be produced and then screened against other protein targets of interest to determine which proteins interact. Randomly modified antibodies or randomly produced peptide fragments can be screened against known proteins of interest to determine which modified antibodies or random peptide fragments bind to the proteins of interest. Another application of the current invention is identifying DNA-DNA, RNA-RNA, or DNA-RNA, DNA-PNA (Peptide Nucleic Acid), and RNA-PNA interactions. Nucleic acids are good candidates for this method because they exhibit many specificities and can be attached to glass slides, beads, and other surfaces with methods known to those skilled in the art. Yet another application of the invention is identifying interactions between cells and proteins, nucleic acids, lipids, chemical compounds, and other particles. Flow cytometry can be used to detect cells that bind to labeled particles. The cells can be identified by a label of their own or can be identified after the initial step of identifying which cells interacted with the labeled particle, for example, one could identify the cell's DNA, size, or its ability to bind to an antibody.

In view of the above explanation of the applications of the invention, it should be understood that although the following is not to be considered limiting, particularly useful implementations of the present methods involve two or more groups of particles in which about one hundred or more particle types are present in each group.

Although the invention has been described with particularity above, it is to be considered limited only insofar as is set forth in the accompanying claims.

The invention claimed is:

1. A method for creating labeled particles, comprising creating at least one mixture containing at least two particle types labeled with at least two label types.

2. A method for assessing particle interactions among a plurality of particles distributed among at least two groups, comprising:
selecting a first group containing particles of at least two particle types;
selecting a second group containing particles of at least two particle types;
labeling at least some of the particles with one or more label types;
combining samples from each group according to the equation
Z=y(LogₓA)
wherein:
Z is the minimum number of group sample combinations to be combined, rounded up to a whole number if necessary,
A is the largest number of particle types in any group,
x is the number of label types,
y is 0.1 to 1.0,
provided, however, that when x 1, Z will equal 2; and
determining, by observation of particle interactions from the group sample combinations, the overall particle interactions from among said groups.

3. The method of claim 2, wherein said label types are selected from the group consisting offluorescence, luminescence, organic dyes, inorganic dyes, radioactive isotopes, quantum dots, DNA, antigens, antibodies, particles that emit radio frequency signals, and beads.

4. The method of claim 2, wherein said label types include secondary particles selected from the group consisting of antibodies, antigens, nucleic acid molecules, and beads, with said secondary particles being labeled with a label type selected from the group consisting of fluorescence, luminescence, organic dyes, inorganic dyes, radioactive isotopes, quantum dots, DNA, antigens, antibodies, particles that emit radio frequency signals, and beads.

5. The method of claim 2, wherein at least 10% of said combinations, but not less than two, are free from degenerative patterns.

6. The method of claim 2, further comprising labeling at least some of the particles with two or more label types.

7. The method of claim 2, further comprising selecting a first group containing particles of at least three particle types and selecting a second group containing particles of at least three particle types.

8. The method of claim 2, further comprising labeling at least some of the particles with three or more label types.

9. The method of claim 2, wherein said groups differ by at least one particle type.

10. The method of claim 2, wherein said groups contain substantially the same particle types.

11. The method of claim 2, wherein said groups differ in concentration of at least one particle type.

12. The method of claim 2, wherein said observation is conducted with a means of detection selected from the group consisting of a flow cytometer, a luminometer, a fluorometer, photography, a digital camera, and photocells.

13. The method of claim 2, wherein said combining is performed spatially by separating said second set of particle types of said grouping into known locations.

14. The method of claim 2, wherein said combining is performed by attaching the particles of one of the groups to a solid surface as sorted by particle type.

15. The method of claim 2, wherein said combining is performed by segregating the particles of one of the groups, as sorted by particle type, by means of at least one physical barrier on a solid surface.

16. The method of claim 2, wherein said combining is performed by segregating the particles of one of the groups, as sorted by particle type, by means of at least one physical barrier wherein said barrier is achieved by enclosing said particles in a structure.

17. The method of claim 2, wherein the number of sample combinations can be increased above the minimum number defined by the equation Z=y(LogₓA) to provide error correction yet to maintain the total number of combinations significantly lower than if simplex experiments were the basis of the combinations.

18. The method of claim 2, wherein the determining step is performed by a computer.

19. A database created according to the method of claim 18.
